(19) United States
(12) Patent Application Publication
(10) Pub. No.: US 2013/0101532 A1
(43) Pub. Date: Apr. 25, 2013

(54) SYNERGISTIC UVR ABSORBING ADJUVANT COMBINATION

(71) Applicants: LAURA A. SPAULDING, Wayne, NJ (US); Patricia L. Scott, Union, NJ (US); Alissa R. Frontauria, Lodi, NJ (US)

(72) Inventors: LAURA A. SPAULDING, Wayne, NJ (US); Patricia L. Scott, Union, NJ (US); Alissa R. Frontauria, Lodi, NJ (US)

(21) Appl. No.: 13/452,458

(22) Filed: Apr. 20, 2012

Related U.S. Application Data
(60) Provisional application No. 61/477,903, filed on Apr. 21, 2011.

(51) Int. Cl. A61K 8/90; A61K 8/37; A61K 8/46; A61K 8/40; A61K 8/92

(52) U.S. Cl. CPC A61K 8/90; A61K 8/40; A61K 8/92; A61K 8/466; A61K 8/37
USPC 424/60; 514/785; 514/772.1; 424/59

(57) ABSTRACT

A composition comprising ethylhexyl methoxycrylene, a compound with multiple phenyl groups, and a carrier oil is provided. The composition may also include an organic sunscreen. The combination exhibits an unexpected synergistic effect, in that the photoactive properties of the composition (e.g., SPF, UV absorption) are significantly higher than would be predicted based on the values for each of the components individually.

### CROSS-REFERENCE TO RELATED APPLICATIONS

The present application claims the benefit of U.S. Provisional Patent Application No. 61/477,903, filed on Apr. 21, 2011.

### BACKGROUND OF THE DISCLOSURE

1. Field of the Disclosure

The present disclosure relates to enhancing the photoactivity of sunscreen adjuvants, such as the sun protection factor (SPF), and/or the UVA (ultraviolet-A) and/or UVB (ultraviolet-B) absorption. In particular, the present disclosure relates to enhancing these properties by placing ethylhexyl methoxycrylene, one or more compounds having multiple phenyl groups, and one or more carrier oils in a composition, and/or subsequently placing them in photoprotective compositions.

2. Description of the Related Art

It is always a goal in the field of suncare to either use less sunscreen active materials while maintaining a desired level of SPF and/or UVA absorption, or to achieve a very high SPF and/or UVA absorption rate overall. Thus, there is a need to enhance the photoactivity of sunscreen adjuvants, namely by boosting the SPF, and/or UVA absorption of materials used in sunscreen compositions.

### SUMMARY OF THE DISCLOSURE

The present disclosure provides a composition comprising ethylhexyl methoxycrylene or derivatives thereof, one or more compounds having multiple phenyl groups, and one or more carrier oils. The composition exhibits a synergistic effect, in that the photoactivity of the composition is greater than what would be expected based on the photoactivity of each of the ingredients individually.

In one embodiment, the present disclosure provides a composition comprising 0.1 wt % to 6.0 wt % of ethylhexyl methoxycrylene, a derivative thereof, or a combination of the two, 0.1 wt % to 4.0 wt % of a compound having multiple phenyl groups selected from the group consisting of benzene sulfonic acids, salts of benzene sulfonic acids, styrenic block copolymers with a hydrogenated midblock of styrene-ethylene/butylene-styrene, styrenic block copolymers with a hydrogenated midblock of styrene-ethylene/propylene-styrene, styrene/butadiene/styrene block copolymers, styrene/isoprene/styrene block copolymers, ethylene/butadiene/styrene block copolymer, ethylene/propylene/styrene block copolymer, styrene/ethylene/butadiene block copolymer, styrene/propylene/butadiene block copolymer, and derivatives or combinations thereof, and a carrier oil.

In another embodiment, the present disclosure provides a composition comprising 0.25 wt % to 2.0 wt % of ethylhexyl methoxycrylene, a derivative thereof, or a combination of the two, 0.2 wt % to 1.0 wt % of a compound having multiple phenyl groups selected from the group consisting of benzene sulfonic acids, salts of benzene sulfonic acids, styrene/ethylene/butadiene block copolymer, and combinations thereof, and a carrier oil selected from the group consisting of mineral oil, isopropyl myristate, butyloctyl salicyclate, ethylhexyl salicyclate, and any combinations thereof.

### BRIEF DESCRIPTION OF THE DRAWINGS

FIG. 1 shows a theoretical diagram of a complex formed between ethylhexyl methoxycrylene and a compound having multiple phenyl compounds in a composition of the present disclosure; and

FIGS. 2-5 depict graphs that illustrate the photoactivity of the compositions of the present disclosure, and are discussed in greater detail below.

### DETAILED DESCRIPTION OF THE DISCLOSURE

The present disclosure provides compositions (e.g., dispersions and emulsions) that enhance the photoactivity of certain adjuvants. In particular, the present disclosure has discovered that when ethylhexyl methoxycrylene is combined with one or more compounds having multiple phenyl groups and one or more carrier oils, the photoactivity of each is greatly enhanced. This is a very surprising result, since, as discussed in greater detail below, the SPF and the UVA/UVB/UVA1 absorption for the combination of the three elements were much greater than one would expect based on the corresponding values for each component individually. The term “photoactivity” is defined as the ability of a compound to attenuate or absorb ultraviolet radiation (UVR). The photoactivity of a compound can refer to, but is not limited to, its SPF, or UVA, UVB, and UVA1 absorption characteristics.

Ethylhexyl methoxycrylene (EHM), a derivative thereof, or a combination of the two can be used in the compositions of the present disclosure. EHM is sold under the trade name Solastay® S1 by Hallstar. EHM can be present in an amount of 0.1-10.0% w/w, 0.1-6.0% w/w, 0.25-2.0% w/w, or 0.5-2.0% w/w, based on the total weight of the emulsion or other type of photoprotective composition, or any subranges in between.

The compounds having multiple phenyl groups can be selected from: benzene sulfonic acids or salts thereof, styrenic block copolymers with a hydrogenated midblock of styrene-ethylene/butylene-styrene (SEBS), styrenic block copolymers with a hydrogenated midblock of styrene-ethylene/propylene-styrene (SEPS), styrene/butadiene/styrene (SBS) block copolymers, styrene/isoprene/styrene (SIS) block copolymers, an ethylene/butadiene/styrene (EBS) block copolymer, an ethylene/propylene/styrene (EPS) block copolymer, styrene/ethylene/butadiene (SEB) block copolymer, styrene/propylene/butadiene (SPB) block copolymer, and any derivatives, or any combinations thereof. Examples of the SEBS, SEPS, SBS, SIS, EBS, EPS, SEB, and SPB block copolymers are the Kraton® D and Kraton® G series from Kraton Polymers. An example of a benzene sulfonic acid salt is sodium polystyrene benzene sulfonate (available, for example, as Flexan® II, from AzkoNobel).

In one embodiment, the compound having multiple phenyl groups is the SEB block copolymer, sold under the trade name Kraton® G1650. Other suitable Kraton® polymers include D1164 PT, and G1702 HU. The compound having multiple phenyl groups can be present in an amount of 0.1-4.0% w/w, 0.20-1.0% w/w, or 0.1-0.5% w/w, based on the total weight of the emulsion or other type of photoprotective composition, or any subranges in between.

The carrier oils used in this disclosure can be those with low polarity that do not exhibit meaningful SPF on their own, such as mineral oil and isopropyl myristate. The carrier oils can also be those with comparatively higher polarity and measurable SPF, such as butyloctyl salicyclate and ethylhexyl salicyclate, the latter of which is also known as octisalate. In general, carrier oils can be aromatic and/or non-aromatic esters, and aromatic and/or nonaromatic hydrocarbon liquids. Nonaromatic versions can include straight and/or branched hydrocarbon chains, saturated and/or unsaturated hydrocarbon chains.

The amount of carrier oil used in the composition will depend on the amounts of the other ingredients. In one embodiment, the amount of carrier oil that will be present in the composition is the remainder after any or all of the ingredients above are incorporated into the composition. The amount of carrier oil may also be such that another carrier may be used, such as water. In one embodiment, the carrier oil is present in an amount of 5-95% w/w, based on the total weight of the emulsion or other type of photoprotective composition, or any subranges in between.

Table I below shows data that examines the effect of various compounds on the photoactivity properties in-vitro SPF and UVA1 absorption. The initials “OS” refer to octisalate, “SEB/OS” to the SEB polymer with octisalate, “IPM” is isopropyl myristate, and “EHM” is ethylhexyl methoxycrylene.

TABLE I
Synergistic UVR Absorption Effect of SEB Polymer/EHM
Dispersions in OS

| | Ingredient Ratio | | | Absorption Response | |
|---|---|---|---|---|---|
| | | SEB/ | | in-vitro SPF | UVA1 |
| Sample ID | OS | OS | EHM | (units) | λnm@0.5 abs |
| 3043-3-1 | 4.0 | — | — | 14 ± 0 | 337.5 |
| 3043-3-2 | — | 4.0 | — | 14 + 0 | 336.5 |
| 3043-3-14 | 4.0 | | 0.4 | 21 ± 3 | 380.0 |
| | (IPM) | | | | |
| 3043-3-4 | — | 4.0 | 0.4 | 106 ± 4 | 382.0 |

As shown in Table I, octisalate exhibits measurable in-vitro SPF (14 units). The SEB polymer does not contribute to the in-vitro SPF when combined with octisalate, as the combination (K/OS) exhibits the same in-vitro SPF, 14 units. The EHM on its own exhibits an in-vitro SPF of 21 units. (In sample 3043-3-14, EHM was combined with isopropyl myristate instead of octisalate, which as stated above does not show any SPF on its own. This was done to get an even clearer idea of the contribution EHM makes to the exhibited synergistic effect.). Thus, upon combining EHM and the SEB polymer, one would expect the resulting composition to have an in-vitro SPF of 35. However, as shown, the synergistic effect on the in-vitro SPF exhibited when the two were combined was over three times that amount (106 units). This result is completely unexpected based on the in-vitro SPF values of the individual components.

EHM can be used to photostabilize sunscreens, such as octinoxate and avobenzone and, as discussed above, has some ability on its own to attenuate UVR. EHM is highly viscous, with strong molecular cohesion forces amongst its molecules. The SEB polymer contains a rubber block portion that includes ethylene and/or propylene and/or isoprene, and butadiene. The SEB polymer has gelling and film-forming capabilities, but not UVR attenuation capabilities. Thus, the results shown in Table I were completely unexpected.

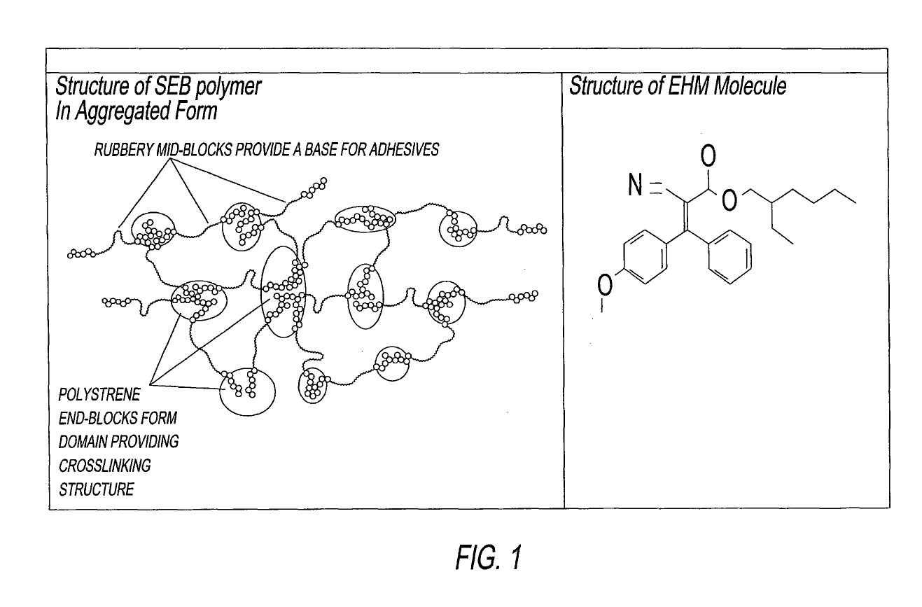

Without being bound by theory, it is proposed that the synergistic effect may be attributed to a lowest free energy complex formed via anchoring the EHM molecule into the interstices of the aggregated phenyl compounds (in this case the SEB polymer), as shown in FIG. 1. The complex formation would augment UVR absorption via the combination of several mechanisms. First, the cohesive forces between EHM molecules would be reduced by dilution in the carrier oil and the SEB polymer, thereby allowing a more uniform film of EHM molecules as they occupy more of the interstices in the SEB polymer. Second, the polystyrene domains of the SEB polymer, in close proximity to the EHM phenyl groups, may act as an electron sink so that EHB molecules can more efficiently absorb UV energy and dissipate the excited photon energy. Lastly, there may be a preferred, strong attraction between the polystyrene domains of the SEB polymer and the phenyl groups of EHM, so that a lowest free energy complex is formed and does not separate.

The compositions of the present disclosure can be further used in conjunction with compositions containing organic sunscreens, as the compositions of the present disclosure may also enhance the photoactivity of those sunscreens as well. Suitable organic sunscreens may include, but are not limited to, cinnamates, octisalate, p-aminobenzoic acid, its salts and its derivatives (ethyl, isobutyl, glyceryl esters; p-dimethylaminobenzoic acid); anthranilates (o-aminobenzoates; methyl, menthyl, phenyl, benzyl, phenylethyl, linalyl, terpinyl, and cyclohexenyl esters), salicylates (octyl, amyl, phenyl, benzyl, menthyl (homosalate), glyceryl, and dipropyleneglycol esters), cinnamic acid derivatives (menthyl and benzyl esters, alpha-phenyl cinnamonitrile; butyl cinnamoyl pyruvate), dihydroxycinnamic acid derivatives (umbelliferone, methylumbelliferone, methylaceto-umbelliferone), camphor derivatives (3-benzylidene, 4-methylbenzylidene, polyacrylamidomethyl benzylidene, benzalkonium methosulfate, benzylidene camphor sulfonic acid, and terephthalylidene dicamphor sulfonic acid), trihydroxycinnamic acid derivatives (esculetin, methylesculetin, daphnetin, and the glucosides, esculin and daphnin), hydrocarbons (diphenylbutadiene, stilbene), dibenzalacetone and benzalacetophenone, naptholsulfonates (sodium salts of 2-naphthol-3,6-disulfonic and of 2-naphthol-6,8-disulfonic acids), dihydroxy-naphthoic acid and its salts, o- and p-hydroxydiphenyldisulfonates, coumarin derivatives (7-hydroxy, 7-methyl, 3-phenyl), diazoles (2-acetyl-3-bromoindazole, phenyl benzoxazole, methyl naphthoxazole, various aryl benzothiazoles), quinine salts (bisulfate, sulfate, chloride, oleate, and tannate), quinoline derivatives (8-hydroxyquinoline salts, 2-phenylquinoline), hydroxy- or methoxy-substituted benzophenones, uric and vilouric acids, tannic acid and its derivatives, hydroquinone, benzophenones (oxybenzone, sulisobenzone, dioxybenzone, benzoresorcinol, 2,2′,4,4′-tetrahydroxybenzophenone, 2,2′-dihydroxy-4,4′-dimethoxybenzophenone, octabenzone), dibenzoylmethane derivatives, avobenzone, 4-isopropyldibenzoylmethane, butylmethoxydibenzoylmethane, 4-isopropyl-dibenzoylmethane, octocrylene, drometrizole trisiloxane, bemotrizinol (sold under the trade name Tinosorb®), ecamsule (sold under the trade name Mexoryl®), and any combinations thereof.

The data below further illustrates the novel and synergistic effects of the emulsion of the present disclosure.

### Materials Used

SEB SEB polymer (e.g., Kraton® G1650)
EHM Ethylhexyl methoxycrylene (e.g., Solastay® S1)

### MO Mineral Oil

### IPM Isopropyl Myristate

### BHB Butyloctyl Salicylate

### OS Ethylhexyl Salicylate

### Dispersions Used

SEB/MO 5% SEB polymer in Isopropyl Myristate (IPM
SEB/IPM 5% SEB polymer in Isopropyl Myristate (IPM)
SEB/BHB 5% SEB polymer in Butyloctyl Salicylate (BHB)
SEB/OS 5% SEB polymer in Ethylhexyl Salicylate (OS)

### Instrumentation

The in-vitro SPF was determined using the Labsphere 2000S UV Transmittance Analyzer and PMMA roughened surface plates. The Labsphere uses a xenon flashlamp supplying sufficient energy for the spectral range of 250-450 nm.

Solvent polarity was determined using the Scientifica 870 Liquid Dielectric Constant Meter.

### Study I

Two sets of samples were needed to characterize a UVR absorption profile for EHM, and establish baselines for in-vitro SPF (magnitude) and UVA1 absorbance at λnm@0.5 abs (breadth). Table II summarizes samples with EHM dispersions in a relatively low polar, UVR transparent solvent (isopropyl myristate, 3.25 dielectric constant) without (control sample) and with (test sample) the SEB polymer. The in-vitro SPF and UVA1 absorbance at λnm@0.5 abs values were recorded to help characterize the UVR absorption profile.

TABLE II
Synergistic UVR Absorption Effect of SEB Polymer/EHM
Dispersions in IPM

| | Ingredient Ratio | | | Absorption Response | |
|---|---|---|---|---|---|
| | | SEB/ | | in-vitro SPF | UVA1 |
| Sample ID | IPM | IPM | EHM | (units) | λnm@0.5 abs |
| 0031-17-25 | 5.0 | — | — | 1 ± 0 STD | — |
| 0031-18-25 | 4.5 | — | 0.5 | 13 ± 1 STD | 374 |
| 0031-17-26 | — | 5.0 | — | 1 ± 0 STD | — |
| 0031-18-26 | — | 4.5 | 0.5 | 22 ± 1 STD | 377 |

Dose: 0.55 mg/cm2

The data in Table II shows that the in-vitro SPF contribution of 10% EHM in low polarity IPM solvent was 13 units. The presence of the SEB polymer, a polar material, unexpectedly boosted the UVR absorption efficacy of EHM to an in-vitro SPF value of 22 units. This is quite unexpected because the SEB polymer/IPM dispersion is essentially transparent to UVR. The +9 in-vitro SPF unit difference is surprisingly significant, because theoretically there should not have been any increase in in-vitro SPF magnitude. Not only did the UVR absorption magnitude increase, but the breadth of UV absorption increased as well, from 374 nm to 377 nm. The observed absorption response values that characterized the synergy were even more surprisingly unexpected because the sample dosing at 0.55 mg/cm², which was well below the FDA recommended dosing of 2 mg/cm², showed a measurable effect in magnitude and breadth of absorption.

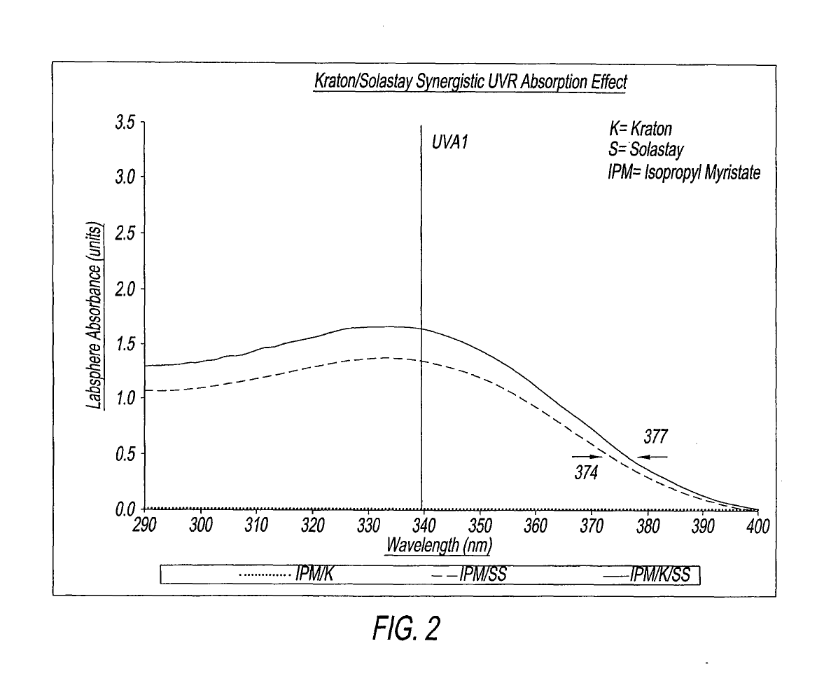

The UV absorption profiles in FIG. 2 illustrate the data shown above in Table II with respect to the UVA1 absorption characteristics.

### Study II

This study characterized the interaction of ethylhexyl salicylate with EHM and the SEB polymer, dosed at 0.60 mg/cm². Ethylhexyl salicylate is also known as octisalate, a relatively polar diluent with a dielectric constant of 6.25, and has UV absorbing capabilities. The first four samples in Table III represent control samples to characterize the interaction of octisalate with EHM. The trend in absorption responses was unexpected. There were significant increases in both in-vitro SPF magnitude and UVA1 breadth, even as the octisalate content decreased and the EHM content increased. The 10% EHM/OS sample (0031-18-31) had an in-vitro SPF of 65 units, as compared to the 13 units for the 10% EHM/IPM sample (0031-18-25) shown above in Table II. This is a five-fold increase in efficacy, and is again much more than one would expect based on the SPF value for octisalate alone. The 3 nm increase in UVA1 λnm@0.5 abs for the same comparison also implied increased absorption efficacy.

TABLE III
Synergistic UVR Absorption Effect of SEB Polymer/EHM
Dispersions in OS

| | Ingredient Ratio | | | Absorption Response | |
|---|---|---|---|---|---|
| | | SEB/ | | in-vitro SPF | UVA1 |
| Sample ID | OS | OS | EHM | (units) | λnm@0.5 abs |
| 0031-17-1 | 5.0 | — | — | 12 + 0 STD | — |
| 0008-88-1 | 4.8 | — | 0.2 | 29 ± 0 STD | 362 |
| 0031-18-31 | 4.5 | — | 0.5 | 65 ± 2 STD | 377 |
| 0008-88-3 | 4.0 | — | 1.0 | 118 ± 3 STD | 388 |
| 0031-17-2 | — | 5.0 | — | 12 ± 0 STD | — |
| 0008-88-2 | — | 4.8 | 0.2 | 39 ± 1 STD | 366 |
| 0031-18-32 | — | 4.5 | 0.5 | 80 ± 1 STD | 379.5 |
| 0008-88-4 | — | 4.0 | 1.0 | 173 ± 3 STD | 389 |

Dose: 0.55 mg/cm²

The last four samples in Table III contained test samples where the concentration of the SEB/OS blend decreased as the EHM content increased. It should be noted that the presence of 5% SEB polymer in octisalate depressed the dielectric constant of octisalate from 6.25 to 5.98, thus signifying that SEB polymer reduced the polarity of the system. Despite this handicap, the data for in-vitro SPF and UVA1 were unexpectedly higher than the control data set. This phenomenon was surprising because SEB polymer had essentially no boosting effect on octisalate absorption efficacy, as illustrated by the fact that samples 0031-17-1 and 0031-17-2 both exhibited an in-vitro SPF of 12 units. The in-vitro SPF response of 80 units for the test sample for 10% EHM in SEB/OS (sample 0031-18-32) versus the in-vitro SPF response of 65 units for the control sample for 10% EHM in OS (sample 0031-18-31) suggested a synergistic action took place between SEB polymer and EHM. The same trend is noted for the 20% EHM in SEB/OS dispersion (sample 0008-88-4) versus the non-SEB polymer containing dispersion (sample 0008-88-3).

### Study III

The UVR absorption synergy with the SEB polymer/EHM combination was found to be further augmented with solvents of increasing polarity. Table IV summarizes the dielectric constants of a group of solvents that were also selected based on their ability to absorb UVR.

TABLE IV
Solvent Polarity

| | Dielectric | UVR |
|---|---|---|
| Solvent | Constant | Absorptive |
| Mineral Oil | 2.10 | No |
| Isopropyl Myristate | 3.25 | No |
| Butyloctyl Salicylate | 5.27 | Yes |
| Ethylhexyl Salicylate | 6.25 | Yes |

Dose: 0.55 mgm/cm2

The first four samples in Table V below are the control samples containing no SEB polymer. The absorption response data for EHM dispersed in UVR transparent and UVR absorbing solvents suggested that even small increases in solvent polarity positively affect EHM's ability to attenuate UVR. The second data set included SEB polymer as well as EHM dispersed in UVR transparent and UVR absorbing solvents. The values of the UVR absorption response data showed a significant increase in both in-vitro SPF magnitude and UVA1 breadth. With careful selection of polar solvent and the combination of SEB polymer and EHM, the in-vitro SPF magnitude can increase by approximately 70 SPF units, and the breadth of UVA1 absorption can increase by approximately 5.5 nm.

TABLE V
Synergistic UVR Absorption Effect of SEB Polymer/EHM
Dispersions in OS

| | Ingredient Ratio | | | Absorption Response | |
|---|---|---|---|---|---|
| | | SEB/ | | in-vitro | |
| | Solvent | Solvent | | SPF | UVA1 |
| Sample ID | @4.5 | @4.5 | EHM | (units) | λnm@0.5 abs |
| 0008-91-1 | MO | — | 0.5 | 7 ± 0 STD | 373.5 |
| 0031-18-25 | IPM | — | 0.5 | 13 ± 1 STD | 374 |
| 0008-91-2 | BHB | — | 0.5 | 56 ± 2 STD | 376 |
| 0031-18-31 | OS | — | 0.5 | 65 ± 2 STD | 377 |
| 0008-91-3 | — | MO | 0.5 | 14 ± 1 STD | 374 |
| 0031-18-26 | — | IPM | 0.5 | 22 ± 1 STD | 377 |
| 0008-91-4 | — | BHB | 0.5 | 67 ± 1 STD | 379 |
| 0031-18-32 | — | OS | 0.5 | 80 ± 1 STD | 379.5 |

Dose: 0.55 mgm/cm2

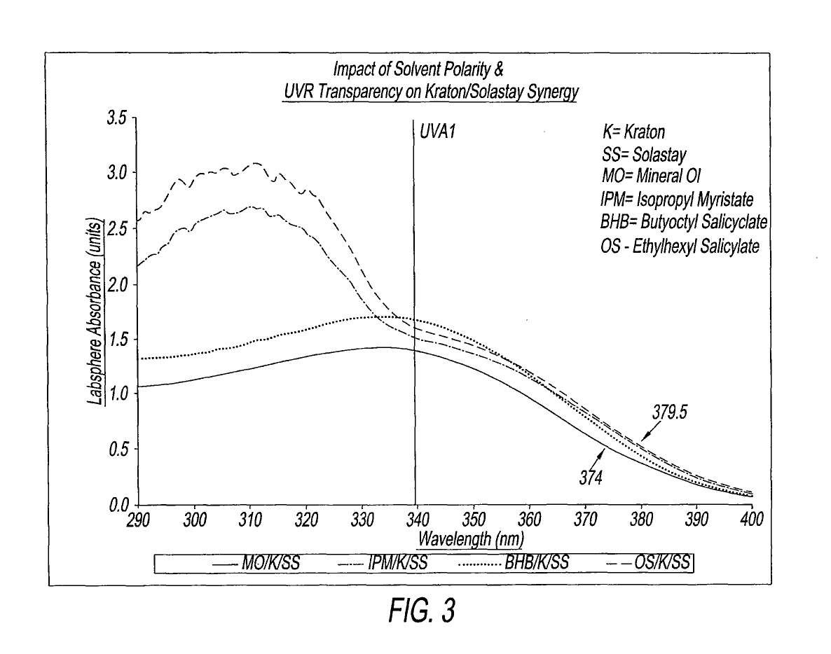

The trends presented in this study also confirmed the synergistic interaction between SEB polymer and EHM to attenuate UVA and UVB rays, as depicted in FIG. 3.

### Study IV

The purpose of this particular study was to examine the effect of a combination of SEB polymer and EHM on the absorption efficacy of avobenzone. In order to study cause and effect of one variable at a time and stay within the dynamic range of the Labsphere instrument, isopropyl myristate was chosen as the q.s. diluent due to its low polarity and transparency to UVR. The first four samples in Table VI represent the control samples, whereby avobenzone content remained constant, EHM content increased, and SEB polymer was absent. The absorption response data clearly demonstrated the positive influence 5% and 10% EHM had on increasing in-vitro SPF and UVA1.

TABLE VI
Synergistic UVR Absorption Effect
of SEB Polymer/EHM Dispersions on Avobenzone

| | | | | | | Absorption Response | |
|---|---|---|---|---|---|---|---|
| | Ingredient Ratio | | | | | in-vitro SPF | UVA1 |
| Sample ID | IPM | OS | SEB/OS | EHM | Avo | (units) | λnm@0.5 abs |
| 0008-90-7 | 5.00 | 5.0 | — | — | | 9 ± 0 STD | — |
| 0008-91-5 | 4.00 | 5.0 | — | — | 1.0 | 24 ± 2 STD | 388.5 |
| 0008-90-8 | 3.75 | 5.0 | — | 0.25 | 1.0 | 35 ± 2 STD | 389 |
| 0008-90-9 | 3.50 | 5.0 | — | 0.50 | 1.0 | 117 ± 3 STD | 392 |
| 0008-90-10 | 5.00 | — | 5.0 | — | — | 11 ± 0 STD | — |
| 0008-91-6 | 4.00 | — | 5.0 | — | 1.0 | 75 ± 3 STD | 391 |
| 0008-90-11 | 3.75 | — | 5.0 | 0.25 | 1.0 | 94 ± 3 STD | 391 |
| 0008-90-12 | 3.50 | — | 5.0 | 0.50 | 1.0 | 156 ± 2 STD | 393 |

Dose: 0.38 mgm/cm2

The last four samples in Table VI clearly show the positive, synergistic influence SEB polymer had on boosting EHM UVR absorption capability. The presence of SEB polymer had essentially no effect on octisalate absorption as illustrated by the in-vitro SPF values of 9 (sample 0008-90-7) and 11 (sample 0008-90-10). However, SEB polymer at a 1:10 and 1:20 ratio with EHM significantly boosted EHM's ability to absorb in-vitro SPF from 35 to 94 units and 117 to 156 units, respectively. The sample containing no SEB polymer/EHM (#0008-91-5) versus the sample with SEB polymer/EHM (#0008-90-12) showed an impressive 6.5 fold increase in in-vitro SPF as the magnitude increased from 24 to 156 units.

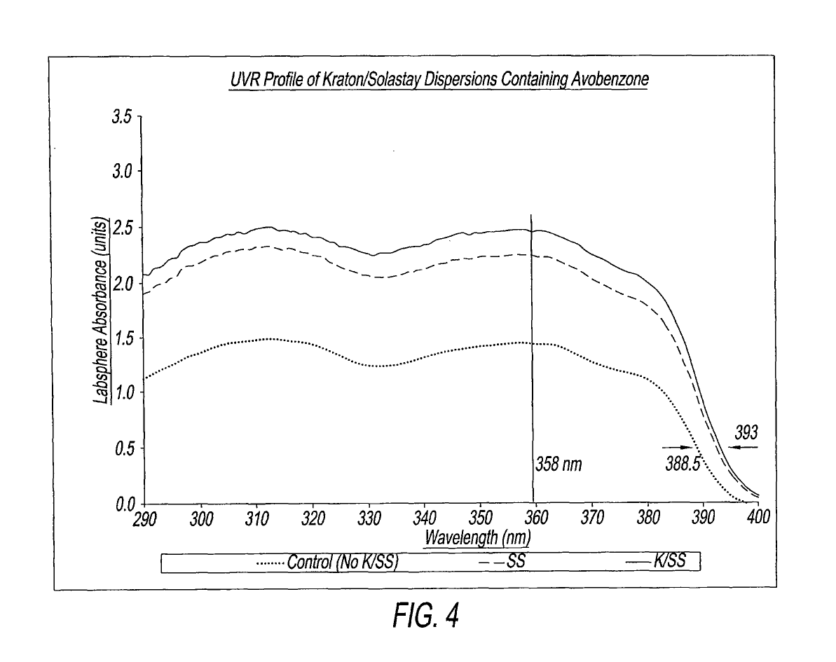

According to literature the wavelength of maximum absorption for avobenzone is 358 nm. Inspection of UVA1 absorption maxima data for the control sample containing is no SEB polymer/EHM versus the test sample with SEB polymer/EHM indicated no shifting or peak broadening at or around 358 nm as shown in FIG. 4. Therefore, the increases noted in absorption responses are due to the SEB polymer/EHM combination.

### Study V

This study focused on formulating the SEB polymer/EHM combination into an SPF 30 sunscreen water-in-oil emulsion to verify the absorption responses noted for the oil phases above. A series of typical sunscreen formulations were prepared that included controls and the test samples containing SEB polymer and EHM. All samples were tested for in-vitro absorption responses regarding SPF and UVA1, and in-vivo SPF at an independent laboratory. Table VII summarizes the sunscreen actives content and the measured absorption responses.

TABLE VII
SEB Polymer/EHM Dispersion in Typical Sunscreen O/W Emulsion

| | Batch No.: | | | |
|---|---|---|---|---|
| | 0008-92 | 0008-93 | 0008-94 | 0008-95 |
| Ingredient | % | % | % | % |
| SEB polymer | — | 0.26 | — | 0.26 |
| EHM | — | — | 1.00 | 1.00 |
| Octisalate | 5.00 | 5.00 | 5.00 | 5.00 |
| Homosalate | 10.00 | 10.00 | 10.00 | 10.00 |
| Avobenzone | 2.00 | 2.00 | 2.00 | 2.00 |
| Octocrylene | 2.40 | 2.40 | 2.40 | 2.40 |
| in-vitro SPF | 42 ± 2 STD | 42 ± 2 STD | 66 ± 1 STD | 87 ± 1 STD |
| UVA1 | 390.5 | 390.5 | 392.0 | 393.0 |
| λnm@0.5 abs | | | | |
| in-vivo SPF | 26 ± 1 STD | 27 ± 0 STD | 30 ± 1 STD | 37 ± 1 STD |
| 5 subjects | | | | |

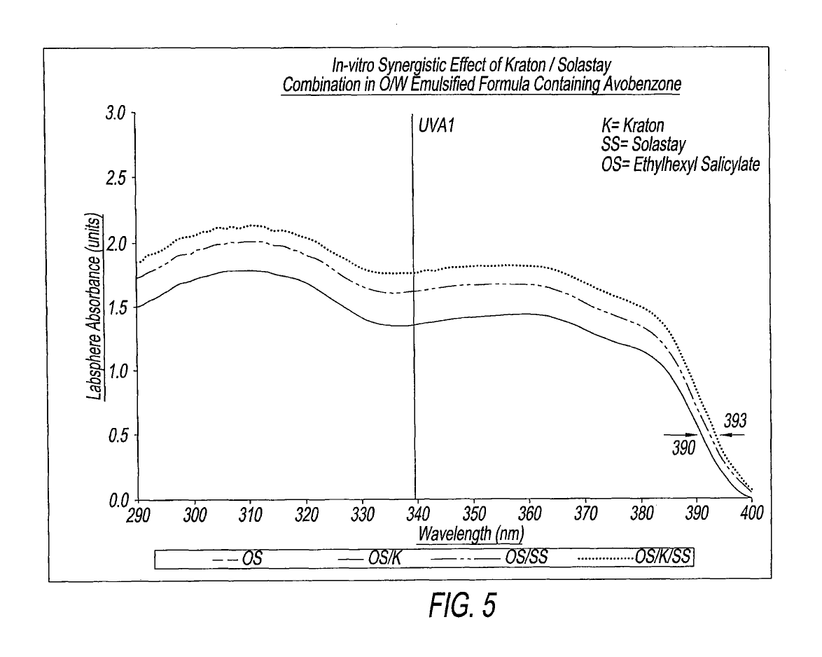

The trend in-vitro and in-vivo SPF for the sunscreen emulsion systems matched those of the oil systems presented above, as shown in FIG. 5.

The presence of SEB polymer without EHM had no influence on absorption responses, as noted earlier. EHM provided some boosting effect as expected. However, the combination of SEB polymer/EHM significantly boosted the in-vitro SPF magnitude, by a 2-fold increase, and the breadth of UVA1 absorption increased by 2.5 nm.

In-vivo SPF results generated by an independent laboratory corroborated the trends noted in the in-vitro studies. The in-vivo SPF for the SEB polymer only formula was essentially comparable to the control formula. The in-vivo SPF for the EHM only formula was significantly higher by 4 SPF units, as expected. But most unexpectedly was the large increase in in-vivo SPF magnitude with the combination of SEB polymer/EHM by 10 SPF units. The in-vivo SPF results support the claim of synergistic UVR boosting activity provided by the combination of SEB polymer/EHM.

### Study VI

The data summarized in Table VIII below demonstrates the synergistic effects of two additional Kraton type materials in the presence of EHM, as well as an additional polymer that acts as a film former/dispersant. This polymer is octyldodecyl-propyl-citrate, and one commercially available example is Cosmosurf® CE100, available from SurfaTech.

Cosmosurf® CE-100 (E0008-085-01) has no real in-vitro SPF value, and EHM (E0008-106) absorbs a small amount of UVR as indicated by the in-vitro SPF value of 4. Examination of the data for a combination of CE-100 and IPM with octisalate shows low in-vitro SPF values of 9 and 10. Addition of Kraton D1164PT or Kraton G1702HU to the is aforementioned combination of CE-100/Octisalate had no effect on in-vitro SPF. What was surprising was the significantly higher SPF obtained when EHM was added to the combination of CE100/Kraton/Octisalate. The combination of 10% EHMC in Octisalate produced an SPF of 65. Unexpectedly, the addition of 10% EHMC to the CE-100/Kraton D1164PT or G1702HU/Octisalate increased the in-vitro SPF to 128 and 136, respectively. These values are highly advantageous in that they represent a 2× increase in UV absorption with almost 50% less octisalate. The numbers also show that Kraton® D1164 and Kraton® G1702 exhibit similar synergistic effects, in addition to the Kraton® 1560 discussed in the data above.

TABLE VIII
Synergy of Other Kratons, EHMC, CE-100 in Oil Phases

| | | | | | | in-vitro |
|---|---|---|---|---|---|---|
| | | | Kraton D | Kraton G | | SPF |
| | EHM | CE-100 | 1164 PT | 1702 HU | OS | 0.55 mg/ |
| Notebook # | (%) | (%) | (%) | (%) | (%) | cm² |
| | | | | | | — |
| 0008-085-01 | x | 100.0 | x | x | x | 1 ± 0 |
| 0008-106 | 100.0 | x | x | x | x | 4 ± 0 |
| 0031-018-31 | 10.0 | x | x | x | 90.0 | 65 ± 2 |
| 0008-090-07 | x | 50.0 | x | x | 50.0 | 9 ± 0 |
| | | (IPM) | | | | |
| 0008-096-13 | x | 50.0 | x | x | 50.0 | 10 ± 1 |
| 0091-041-04 | x | 50.0 | 2.5 | x | 47.5 | 9 ± 0 |
| 0091-042-12 | 2.0 | 48.0 | 2.5 | x | 47.5 | 38 ± 3 |
| 0091-043-20 | 5.0 | 45.0 | 2.5 | x | 47.5 | 77 ± 4 |
| 0091-044-28 | 10.0 | 40.0 | 2.5 | x | 47.5 | 128 ± 1 |
| 0091-054-04 | 10.0 | x | 4.5 | x | 85.5 | 88 ± 0 |
| 0031-018-31 | 10.0 | x | x | x | 90.0 | 65 ± 2 |
| 0091-041-08 | x | 50.0 | x | 2.5 | 47.5 | 8 ± 1 |
| 0091-042-16 | 2.0 | 48.0 | x | 2.5 | 47.5 | 38 ± 3 |
| 0091-043-24 | 5.0 | 45.0 | x | 2.5 | 47.5 | 82 ± 3 |
| 0091-044-32 | 10.0 | 40.0 | x | 2.5 | 47.5 | 136 ± 3 |
| 0091-054-08 | 10.0 | x | 4.5 | x | 85.5 | 88 ± 3 |

The compositions of the present disclosure can take the form of an oil-in-water emulsion, water-in-oil emulsion, or an oily liquid that is not an emulsion. They can also be in the form of a cream, lotion, liquid or stick composition. The data below shows that the above-described synergistic effect is also exhibited in compositions with different forms other than an oil-in-water emulsion.

In Tables IX-XI below, OS=octisalate, HS=homosalate, OC=octocrylene, and AVB=avobenzone.

TABLE IX
Oil-in-Water Emulsion Creme SPF 30
All formulas contain the following additional ingredients: Acrylates C12-22
0.75%, Euxyl PE 9010 or Germall 0.70%, Glycerin 2.00%, Disodium EDTA 0.10%.
Prolipid 141 5.00%, Ceraphyl SLK 5.00%, Ceraphyl 368 4.00%, Squalane 1.00%,
Seppinov EMT10 1.00%, Water q.s. to 100.00%.

| Notebook | | Kraton | | | | | | in-vitro |
|---|---|---|---|---|---|---|---|---|
| Number | CE-100 | 1650G | EHM | OS | HS | OC | AVB | SPF 1 mg/cm² |
| 0057-075 | x | x | x | 5.00% | 10.00% | 2.40% | 2.00% | 93 ± 3 |
| 0057-085 | 3.00% | x | 1.00% | 5.00% | 10.00% | 2.40% | 2.00% | 97 ± 1 |
| 0057-076 | x | 0.26% | 1.00% | 5.00% | 10.00% | 2.40% | 2.00% | 107 ± 1 |
| 0057-107 | x | 0.52% | 3.00% | 5.00% | 10.00% | x | x | 59 ± 2 |
| 0057-109 | 0.50% | 0.26% | 3.00% | 5.00% | 10.00% | x | x | 62 ± 1 |
| 0057-110 | 20.00% | 0.10% | 3.00% | 5.00% | 10.00% | x | x | 65 ± 2 |
| 0057-108 | x | 0.26% | 6.00% | 5.00% | 10.00% | x | x | 80 ± 2 |

TABLE X
Water-in-Oil Emulsion Lotion SPF 30
All formulas contain the following additional ingredients: Dermol M5 10.00%,
Abil EM90 5.00%, Cyclomethicone 3.00%, Abil Wax 9801 1.00%, Emulsynt GDL
2.00%, Vitamin E 0.10%, Butylene Glycol 1.00%, Magnesium Sulfate 0.75%,
Glydant Plus Liquid 0.5%, Water q.s. to 100.00%

| Notebook | | Kraton | | | | | | in-vitro |
|---|---|---|---|---|---|---|---|---|
| Number | CE-100 | 1650G | EHM | OS | HS | OC | AVB | SPF 1 mg/cm² |
| 0057-132 | x | x | x | 5.00% | 10.00% | 2.40% | 2.00% | 127 ± 4 |
| 0057-133 | x | 0.26% | 1.00% | 5.00% | 10.00% | 2.40% | 2.00% | 163 ± 5 |
| 0057-137 | 3.00% | x | 1.00% | 5.00% | 10.00% | 2.40% | 2.00% | 152 ± 1 |
| 0057-136 | 3.00% | 0.26% | 1.00% | 5.00% | 10.00% | 2.40% | 2.00% | 165 ± 3 |
| 0057-148 | x | 0.52% | 3.00% | 5.00% | 10.00% | x | x | 43 ± 2 |
| 0057-150 | 0.50% | 0.26% | 3.00% | 5.00% | 10.00% | x | x | 44 ± 2 |
| 0091-001 | 20.00% | 0.10% | 3.00% | 5.00% | 10.00% | x | x | 42 ± 1 |
| 0057-149 | x | 0.26% | 6.00% | 5.00% | 10.00% | x | x | 91 ± 4 |

TABLE XI
Sunscreen Stick SPF 30
All formulas contain the following additional ingredients: Ozokerite Wax
10.00%, Paraffin Wax 2.00%, Siliconyl Polyethylene 8.00%,
Microcrystalline Wax 5.00%, Propylparaben 0.10%, Si-Tex Wax 2.5%,
Ceraphyl 230 2.00%, Ceraphyl 45 5-10.00%, Si-Tex PTM 20 1.00%,
Emulsynt GDL 7.50%, q.s. Ceraphyl 368 up to 100.00%.

| | | | | | | | | in-vitro |
|---|---|---|---|---|---|---|---|---|
| Notebook | | Kraton | | | | | | SPF 1 |
| Number | CE-100 | 1650G | EHM | OS | HS | OC | AVB | mg/cm² |
| 0057-92 | x | x | x | 5.00% | 10.00% | 2.40% | 2.00% | 75 ± 3 |
| 0091-95 | 3.00% | x | 1.00% | 5.00% | 10.00% | 2.40% | 2.00% | 86 ± 1 |
| 0057-93 | x | 0.26% | 1.00% | 5.00% | 10.00% | 2.40% | 2.00% | 104 ± 2 |
| 0057-94 | 3.00% | 0.26% | 1.00% | 5.00% | 10.00% | 2.40% | 2.00% | 109 ± 2 |
| 0091-023 | x | 0.52% | 3.00% | 5.00% | 10.00% | 2.40% | 2.00% | 135 ± 3 |
| 0091-024 | x | 0.26% | 6.00% | 5.00% | 10.00% | 2.40% | 2.00% | 143 ± 3 |
| 0091-025 | 0.50% | 0.26% | 3.00% | 5.00% | 10.00% | 2.40% | 2.00% | 73 ± 2 |
| 0091-026 | 20.00% | 0.10% | 3.00% | 5.00% | 10.00% | 2.40% | 2.00% | 84 ± 2 |

As is shown in Tables IX-XI, in-vitro SPF data were measured for three additional formulation types—crème, stick and water-in-oil lotions. In general, Kraton 1650G and EHMC synergistically boosted SPF, as in the oil-in-water previously discussed. An optimized ratio of about 1:0.26 EHMC:Kraton would provide the best SPF efficacy results. The addition of Cosmosurf CE-100 further enhanced the in-vitro SPF results. The optimized ratio is about 1:0.26:3 EHMC:Kraton:CE100.

While the present disclosure has been described with reference to one or more particular embodiments, it will be understood by those skilled in the art that various changes may be made and equivalents may be substituted for elements thereof without departing from the scope thereof. In addition, many modifications may be made to adapt a particular situation or material to the teachings of the disclosure without departing from the scope thereof. Therefore, it is intended that the disclosure not be limited to the particular embodiment(s) disclosed as the best mode contemplated for carrying out this disclosure.

What is claimed is:

1. A composition, comprising:
0.1 wt % to 6.0 wt % of ethylhexyl methoxycrylene, a derivative thereof, or a combination of the two;
0.1 wt % to 4.0 wt % of a compound having multiple phenyl groups selected from the group consisting of benzene sulfonic acids, salts of benzene sulfonic acids, styrenic block copolymers with a hydrogenated midblock of styrene-ethylene/butylene-styrene, styrenic block copolymers with a hydrogenated midblock of styrene-ethylene/propylene-styrene, styrene/butadiene/styrene block copolymers, styrene/isoprene/styrene block copolymers, ethylene/butadiene/styrene block copolymer, ethylene/propylene/styrene block copolymer, styrene/ethylene/butadiene block copolymer,
styrene/propylene/butadiene block copolymer, and derivatives or combinations thereof; and
a carrier oil.

2. The composition of claim 1, wherein said compound having multiple phenyl groups is selected from the group consisting of benzene sulfonic acids, salts of benzene sulfonic acids, styrene/ethylene/butadiene block copolymer, and combinations thereof.

3. The composition of claim 1, wherein said compound having multiple phenyl groups is a styrene/ethylene/butadiene block copolymer.

4. The composition of claim 1, wherein said ethylhexyl methoxycrylene is present in an amount of 0.25 wt % to 2.0 wt %.

5. The composition of claim 1, wherein said compound having multiple phenyl groups is present in an amount of 0.2 wt % to 1.0 wt %.

6. The composition of claim 1, wherein said carrier oil is selected from the group consisting of mineral oil, isopropyl myristate, butyloctyl salicyclate, ethylhexyl salicyclate, and any combinations thereof.

7. The composition of claim 1, further comprising water.

8. The composition of claim 7, wherein the composition is an oil-in-water emulsion.

9. The composition of claim 7, wherein the composition is a water-in-oil emulsion.

10. The composition of claim 1, further comprising an organic sunscreen.

11. The composition of claim 10, wherein said organic sunscreen is selected from the group consisting of octisalate, homosalate, octocrylene, avobenzone, and any is combinations thereof.

12. A composition, comprising:
0.25 wt % to 2.0 wt % of ethylhexyl methoxycrylene, a derivative thereof, or a combination of the two;
0.2 wt % to 1.0 wt % of a compound having multiple phenyl groups selected from the group consisting of benzene sulfonic acids, salts of benzene sulfonic acids, styrene/ethylene/butadiene block copolymer, and combinations thereof; and
a carrier oil selected from the group consisting of mineral oil, isopropyl myristate, butyloctyl salicyclate, ethylhexyl salicyclate, and any combinations thereof.

13. The composition of claim 12, wherein said compound having multiple phenyl groups is a styrene/ethylene/butadiene block copolymer.

14. The composition of claim 12, further comprising water.

15. The composition of claim 14, wherein the composition is an oil-in-water emulsion.

16. The composition of claim 14, wherein the composition is a water-in-oil emulsion.

17. The composition of claim 12, further comprising an organic sunscreen.

18. The composition of claim 17, wherein said organic sunscreen is selected from the group consisting of octisalate, homosalate, octocrylene, avobenzone, and any combinations thereof.
